(19) United States
(12) Patent Application Publication
(10) Pub. No.: US 2012/0196793 A1
(43) Pub. Date: Aug. 2, 2012

(54) BIOMARKERS FOR IAP INHIBITOR COMPOUNDS

(76) Inventors: Brant G. Firestone, Cambridge, MA (US); Kymberly Levine, Cambridge, MA (US); Dale A. Porter, Cambridge, MA (US); John Sullivan, Cambridge, MA (US); Leigh Zawel, Cambridge, MA (US)

(21) Appl. No.: 13/496,042

(22) PCT Filed: Sep. 17, 2010
(86) PCT No.: PCT/US10/49207

§ 371 Date: Mar. 14, 2012

Related U.S. Application Data
(60) Provisional application No. 61/243,564, filed on Sep. 18, 2009.

(51) Int. Cl. A61K 38/05; A61P 31/12; A61P 35/00; A61P 9/00; G01N 33/53; C07K 5/078

(52) U.S. Cl. 514/3.7; 435/7.92; 514/21.91; 548/200; 514/19.3; 514/16.4

(57) ABSTRACT

A method to predict which patients will respond to a IAP inhibiting compound comprising administering an IAP inhibitor compound to a patient/and measuring IL1B, Lymphotoxin alpha (LTa), TWEAK, LIGHT, Fas, TNF alpha or TRAIL levels.

### FIELD OF THE DISCLOSURE

The present disclosure relates to a method to predict which patients will respond to a IAP inhibiting compound. Such method comprises administering an IAP inhibitor compound to a patient, and measuring IL1B, Lymphotoxin alpha (LTa), TWEAK, LIGHT, Fas, TNF alpha and/or TRAIL levels.

### BACKGROUND OF THE DISCLOSURE

IAP inhibitor compounds demonstrate single agent activity on a subset of tumor cell lines known to have elevated basal expression levels of the cytokine TNF. The molecular basis for this activity lies in the ability of IAP inhibitor compounds to induce the rapid degradation of CIAP1 protein. CIAP1 normally functions in the TNF signaling cascade to trans-ubiquitinate RIPK, a modification which is essential for NFKB signaling. In the absence of CIAP1, RIPK loses these modifications and TNF becomes potently proapoptotic. TRAIL ligand has also been reported to be a potent synergistic combination partner for IAP Inhibitor compounds. Other than TNF and TRAIL, cytokines which synergize with IAP Inhibitor compounds in killing tumor cells have not been defined.

### SUMMARY OF THE DISCLOSURE

The present disclosure evaluates the entire TNF superfamily as well as additional cytokines for the ability to potentiate IAP inhibitor compound-mediated cell death. The present disclosure, as described herein below overcomes deficiencies in the use of IAP inhibitor compounds by providing a method to determine which individual with a disease characterized by IL1B, Lymphotoxin alpha (LTa), TWEAK, LIGHT, Fas, TNF alpha or TRAIL signaling will respond to treatment with a IAP inhibitor compound.

In another embodiment, the present disclosure relates to the use of compounds that inhibit the binding of the Smac protein to IAP (“IAP inhibitor”) for the treatment of diseases characterized by IL1B, Lymphotoxin alpha (LTa), TWEAK, LIGHT, Fas, TNF alpha or TRAIL signaling, and to a method for the manufacture of a medicament for treating diseases characterized by IL1B, Lymphotoxin alpha (LTa), TWEAK, LIGHT, Fas, TNF alpha or TRAIL signaling, and to a method for the treatment of warm-blooded animals, including humans, wherein an IAP inhibitor is administered to a warm-blooded animal suffering diseases characterized by IL1B, Lymphotoxin alpha (LTa), TWEAK, LIGHT, Fas, TNF alpha or TRAIL signaling, especially proliferative diseases effected by cytokine production such as cancer, arthritis, sepsis, cancer associated cachexia, Crohn's disease and other inflammatory disorders.

In one embodiment, the IAP inhibitor is (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide, also referred to in this application as Compound A.

### DESCRIPTION OF THE FIGURES

FIG. 1 illustrates the impact of treating A2058 melanoma cell with LTa in the presence and absence of Compound A on cellular proliferation.

FIG. 2 illustrates the impact of treating A2058 melanoma cell with TWEAK in the presence and absence of the IAP inhibitor Compound A on cellular proliferation.

FIG. 3 illustrates the impact of treating A2058 melanoma cell with LIGHT in the presence and absence of the IAP inhibitor Compound A on cellular proliferation.

FIG. 4 illustrates the impact of treating A2058 melanoma cell with Fas in the presence and absence of the IAP inhibitor Compound A on cellular proliferation.

FIG. 5 illustrates the impact of treating A2058 melanoma cell with IL-1B in the presence and absence of the IAP inhibitor Compound A on cellular proliferation.

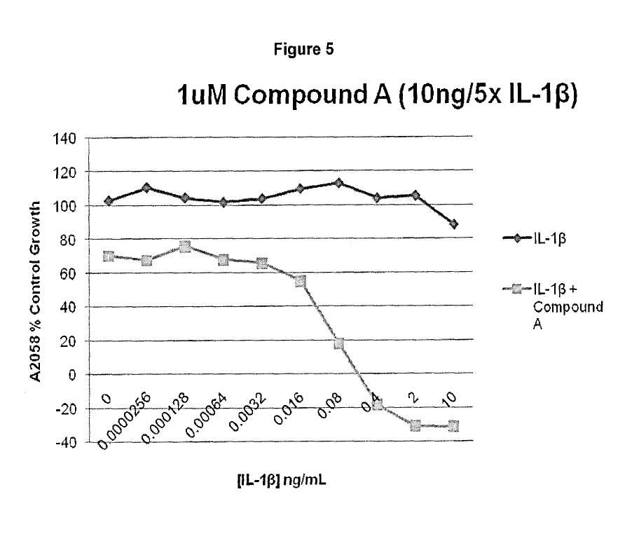

FIG. 6 illustrates the impact of treating A2058 melanoma cell with TRAIL in the presence and absence of the IAP inhibitor Compound A on cellular proliferation.

FIG. 7 illustrates the impact of treating A2058 melanoma cell with TNF alpha in the presence and absence of the IAP inhibitor Compound A on cellular proliferation.

FIG. 8 illustrates that Compound A induced TNF-alpha in sensitive but not insensitive cancer cell lines.

FIG. 9 illustrates that RelA but not RelB is required for Compound A-induced TNFα.

FIG. 10 illustrates that Compound A treatment induced TNFα in breast cancer tumor cell line xenografts.

FIG. 11 illustrates that TNFα expression is correlated with response to Compound A in primary human breast and lung tumor xenografts.

### DETAILED DESCRIPTION OF THE DISCLOSURE

One embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring IL1B, Lymphotoxin alpha (LTa), TWEAK, LIGHT, Fas, TNF alpha and/or TRAIL levels in said patient.

If the level(s) in the patient increases upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least one out of the group consisting of IL1B, Lymphotoxin alpha (LTa), TWEAK, LIGHT, Fas, TNF alpha and TRAIL levels in said patient.

If at least one of the measured level(s) in the patient increases upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least two out of the group consisting of IL1B, Lymphotoxin alpha (LTa), TWEAK, LIGHT, Fas, TNF alpha and TRAIL levels in said patient.

If at least two of the measured levels in the patient increase upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least three out of the group consisting of IL1B, Lymphotoxin alpha (LTa), TWEAK, LIGHT, Fas, TNF alpha and TRAIL levels in said patient.

If at least three of the measured levels in the patient increase upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least four out of the group consisting of IL1B, Lymphotoxin alpha (LTa), TWEAK, LIGHT, Fas, TNF alpha and TRAIL levels in said patient.

If at least four of the measured levels in the patient increase upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least five out of the group consisting of IL1B, Lymphotoxin alpha (LTa), TWEAK, LIGHT, Fas, TNF alpha and TRAIL levels in said patient.

If at least five of the measured levels in the patient increase upon administration of the IAP inhibitor compound, this is an indication that the compound is working.

Another embodiment of this disclosure provides a method to predict which patients will respond to a IAP inhibitor compound in patients having a disease characterized by IL1B, Lymphotoxin alpha (LTa), TWEAK, LIGHT, Fas, TNF alpha or TRAIL signaling comprising:

a) administering an IAP inhibitor compound to a patient, and

b) measuring the level of at least six out of the group consisting of IL1B, Lymphotoxin alpha (LTa), TWEAK, LIGHT, Fas, TNF alpha and TRAIL levels in said patient.

If at least six of the measured levels in the patient increase upon administration of the IAP inhibitor compound, this is an indication that the compound is working.

In another embodiment, the present disclosure relates to the use of compounds that inhibit the binding of the Smac protein to IAPs (“IAP inhibitors”) to manufacture a medicament for the treatment of diseases characterized by constitutive IL1B, Lymphotoxin alpha (LTa), TWEAK, LIGHT, Fas, TNF alpha or TRAIL signaling.

The present disclosure also relates to a method to treat diseases characterized by constitutive IL1B, Lymphotoxin alpha (LTa), TWEAK, LIGHT, Fas, TNF alpha or TRAIL signaling by administering IAP inhibitors in combination with TNF-α, Interferon-alpha or Interferon-gamma or other agents which modulate IL1B, Lymphotoxin alpha (LTa), TWEAK, LIGHT, Fas, TNF alpha or TRAIL signaling.

Examples of IAP inhibitors for use in the present disclosure include compounds of formula I:

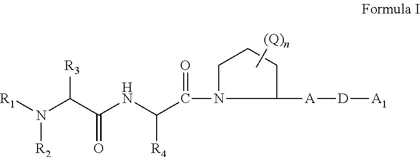

or pharmaceutically acceptable salts thereof, wherein

R₁ is H, C₁-C₄ alkyl, C₂-C₄ alkenyl, C₂-C₄ alkynyl or C₃-C₁₀ cycloalkyl, which R₁ may be unsubstituted or substituted;

R₂ is H, C₁-C₄ alkyl, C₂-C₄ alkenyl, C₂-C₄ alkynyl, C₃-C₁₀ cycloalkyl which R₂ may be unsubstituted or substituted;

R₃ is H, CF₃, C₂F₅, C₂-C₄ alkyl, C₂-C₄ alkenyl, C₂-C₄ alkynyl, CH₂-Z or R₂ and R₃ taken together with the nitrogen atom to which they are attached form a heterocyclic ring, which alkyl, alkenyl, alkynyl or het ring may be unsubstituted or substituted;

Z is H, OH, F, Cl, CH₃, CH₂Cl, CH₂F or CH₂OH;

R₄ is C₀₋₁₀ alkyl, C₃-C₁₀ cycloalkyl, wherein the C₀₋₁₀ alkyl, or cycloalkyl group is unsubstituted or substituted;

A is het, which may be substituted or unsubstituted;

D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C (O) C₀-C₁₀ arylalkyl OC₀-C₁₀ arylalkyl, or S(O)r C₀-C₁₀ arylalkyl, which alkyl and aryl groups may be unsubstituted or substituted;

r is 0, 1, or 2;

A₁ is a substituted aryl or unsubstituted or substituted het which substituents on aryl and het are halo, lower alkoxy, NR₅R₆, CN, NO₂ or SR₅;

each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆-C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂—N(R₁₁)(R₁₂), or NR₁₁—S(O)₂-(R₁₂), wherein alkyl, cycloalkyl and aryl are unsubstituted or substituted;

n is 0, 1,2 or 3, 4, 5, 6 or 7;

het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;

R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂,C(O)-C₁-C₁₀alkyl, —C(O)—CH₂)₁₋₆—-C₃-C₇cycloalkyl, —C(O)—O—(CH₂)₀₋₆-aryl, —C(O)—(CH₂)₀₋₆-O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆-O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)ₘNR₁₁R_{12, m=}1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;

wherein the alkyl substituents of R₁₁ and R₁₂ may be unsubstituted or substituted by one or more substituents selected from C₁-C₁₀alkyl, halogen, OH, O—C₁-C₅alkyl, —S—C₁-C₆alkyl, CF₃ or NR₁₁R₁₂;
substituted cycloalkyl substituents of R₁₁ and R₁₂ are substituted by one or more substituents selected from a C₂-C₁₀alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C_-C₄-alkyl;

R₅, R₆ and R₇ are independently hydrogen, lower alkyl, aryl, aryl lower alkyl, cycloalkyl, or cycloalkyl lower alkyl, and

wherein the substituents on R₁, R₂, R₃, R₄, Q₁ and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N, N-dilower alkyl carbamoyl, mercapto, or lower alkylthio, and

R₉, R₁₀, and R₁₃ are independently hydrogen, lower alkyl, halogen substituted lower alkyl, aryl, aryl lower alkyl, halogen substituted aryl, halogen substituted aryl lower alkyl. Compounds within the scope of formula (I) and the process for their manufacture are disclosed in U.S. 60/835,000, which is hereby incorporated into the present application by reference. The preferred compounds are selected from the group consisting of (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide (Compound II); (S)—N—[(S)-Cyclohexyl-(ethyl-{(S)-1-[5-(4-fluoro-benzoyl)-pyridin-3-yl]-propyl}carbamoyl)-methyl]-2-methylamino-propionamide (Compound III); (S)—N—((S)-1-Cyclohexyl-2-{(S)-2-[5-(4-fluoro-phenoxy)-pyridin-3-yl]-pyrrolidin-1-yl}-2-oxo-ethyl)-2-methylamino-propionamide; and N-[1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

Examples of other IAP inhibitors includes compounds disclosed in WO 05/097791 published on Oct. 20, 2005, which is hereby incorporated into the present application by reference. A preferred compound within the scope of formula (I) is N-[1-cyclohexyl-2-oxo-2-(6-phenethyl-octahydro-pyrrolo[2,3-c]pyridin-1-yl-ethyl]-2-methylamino-propionamide, hereinafter compound II.

Additional IAP inhibitors include compounds disclosed in WO 04/005284, PCT/US2006/013984, PCT/US2006/021850 all of which are hereby incorporated into the present application by reference.

Other IAP inhibitor compounds for use in the present disclosure include those disclosed in WO 06/069063, WO 05/069888, US2006/0014700, WO 04/007529, US2006/0025347, WO 06/010118, WO 05/069894, WO 06/017295, WO 04/007529, WO 05/094818.

In one embodiment, the IAP inhibitor is (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide, also referred to in this application as Compound A.

In each case where citations of patent applications are given above, the subject matter relating to the compounds is hereby incorporated into the present application by reference. Comprised are likewise the pharmaceutical acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the disclosure can be prepared and administered as described in the cited documents, respectively. Also within the scope of this disclosure is the combination of more than two separate active ingredients as set forth above, i.e., a pharmaceutical combination within the scope of this disclosure could include three active ingredients or more.

The terms “treatment” or “therapy” (especially of tyrosine protein kinase dependent diseases or disorders) refer to the prophylactic or preferably therapeutic (including but not limited to palliative, curing, symptom-alleviating, symptom-reducing, kinase-regulating and/or kinase-inhibiting) treatment of said diseases, especially of the diseases mentioned below.

A warm-blooded animal (or patient) is preferably a mammal, especially a human.

Where subsequently or above the term “use” is mentioned (as verb or noun) (relating to the use of an IAP inhibitor), this (if not indicated differently or suggested differently by the context) includes any one or more of the following embodiments of the disclosure, respectively (if not stated otherwise): the use in the treatment of a disease (especially diseases mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling), the use for the manufacture of pharmaceutical compositions for use in the treatment of diseases mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling, methods of use of one or more IAP inhibitors in the treatment of a disease mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling, pharmaceutical preparations comprising one or more IAP inhibitors for the treatment of said disease mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling, and one or more IAP inhibitors in the treatment of said disease mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling, as appropriate and expedient, if not stated otherwise. In particular, diseases to be treated and are thus preferred for “use” of an IAP inhibitor are selected from diseases that are mediated or exacerbated by excessive IL1B, Lymphotoxin alpha (LTa), TWEAK, LIGHT, Fas, TNF alpha or TRAIL or characterized by constitutive IL1B, Lymphotoxin alpha (LTa), TWEAK, LIGHT, Fas, TNF alpha or TRAIL signaling.

Preferred is the use of an IAP inhibitor in the therapy (including prophylaxis) of a proliferative disorder (especially which is characterized by constitutive IL1B, Lymphotoxin alpha (LTa), TWEAK, LIGHT, Fas, TNF alpha or TRAIL signaling) selected from tumor or cancer diseases, especially against preferably a benign or especially malignant tumor or cancer disease, more preferably solid tumors, e.g. carcinoma of the brain, kidney, liver, adrenal gland, bladder, breast, stomach (especially gastric tumors), ovaries, colon, rectum, prostate, pancreas, lung (e.g. small or large cell lung carcinomas), vagina, thyroid, sarcoma, glioblastomas, multiple myeloma (MM) or gastrointestinal cancer, especially colon carcinoma or colorectal adenoma, or a tumor of the neck and head, e.g. squameous carcinoma of the head and neck, including neoplasias, especially of epithelial character, e.g. in the case of mammary carcinoma; an epidermal hyperproliferation (other than cancer), especially psoriasis; prostate hyperplasia; or a leukemia, especially acute myeloid leukemia (AML) and chronic myeloid leukemia (CML).

The precise dosage of an IAP inhibitor compound to be employed depends upon several factors including the host, the nature and the severity of the condition being treated, the mode of administration. The IAP inhibitor compound can be administered by any route including orally, parenterally, e.g., intraperitoneally, intravenously, intramuscularly, subcutaneously, intratumorally, or rectally, or enterally. Preferably the IAP inhibitor compound is administered orally, preferably at a daily dosage of 1-300 mg/kg body weight or, for most larger primates, a daily dosage of 50-5000, preferably 500-3000 mg. A preferred oral daily dosage is 1-75 mg/kg body weight or, for most larger primates, a daily dosage of 10-2000 mg, administered as a single dose or divided into multiple doses, such as twice daily dosing.

Usually, a small dose is administered initially and the dosage is gradually increased until the optimal dosage for the host under treatment is determined. The upper limit of dosage is that imposed by side effects and can be determined by trial for the host being treated.

Dosage regimens must be titrated to the particular indication, the age, weight, and general physical condition of the patient, and the response desired but generally doses will be from about 10 to about 500 mg/day as needed in single or multiple daily administration. In general, an initial treatment regimen can be copied from that known to be effective in interfering with IL1B, Lymphotoxin alpha (LTa), TWEAK, LIGHT, Fas, TNF alpha or TRAIL activity for other IL1B, Lymphotoxin alpha (LTa), TWEAK, LIGHT, Fas, TNF alpha or TRAIL mediated disease states by the compounds of the present disclosure. Treated individuals will be regularly checked for T cell numbers and T4/T8 ratios and/or measures of viremia such as levels of reverse transcriptase or viral proteins, and/or for progression of cytokine-mediated disease associated problems such as cachexia or muscle degeneration. If no effect is soon following the normal treatment regimen, then the amount of cytokine activity interfering agent administered is increased; e.g., by fifty percent a week.

IAP inhibitor compounds may be combined with one or more pharmaceutically acceptable carriers and, optionally, one or more other conventional pharmaceutical adjuvants and administered enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

The following examples are offered by way of illustration and are not intended to limit the scope of the disclosure. The cytokines identified in these examples could potentially be monitored, in plasma and/or in tumor, in the general cancer patient population for the purpose of selecting patients likely to respond to monotherapy using (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide, known as Compound A. This disclosure identifies IL1B, Lymphotoxin alpha (LTa), TWEAK, LIGHT, Fas, TNF alpha and TRAIL as potential companion diagnostic markers for Compound A.

### EXAMPLE 1

Treatment of A2058 melanoma cell with LTa in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and LTa. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted LTa at a final concentration of 10 ng, 5 ng, 2.5 ng, 1.25 ng, 0.625 ng, 312.5 pg, 156.25 pg, 78.125 pg, 39.0625 pg, and one untreated well. Remaining cells are treated with 10 uL of serially diluted LTa and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

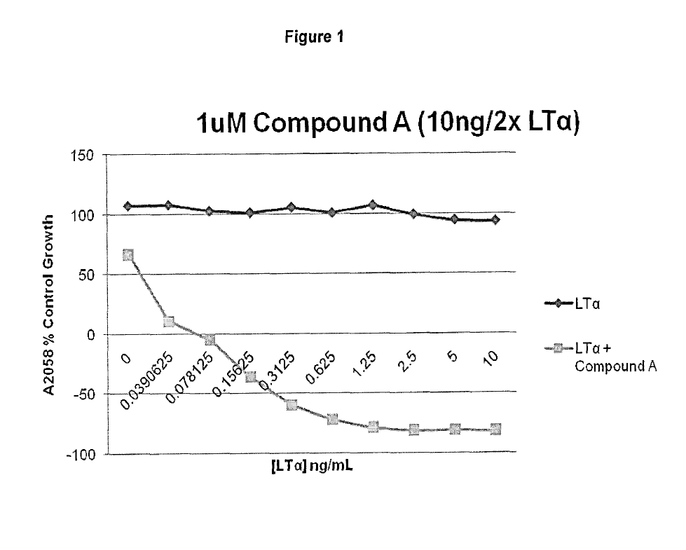

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated in FIG. 1.

### EXAMPLE 2

Treatment of A2058 melanoma cell with TWEAK in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and TWEAK. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted TWEAK at a final concentration of 156.25 ng, 78.125 ng, 39.06 ng, 19.53 ng, 9.77 ng, 4.88 ng, 2.44 ng, 1.22 ng, 610.35 pgng, and one untreated well. Remaining cells are treated with 10 uL of serially diluted TWEAK and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

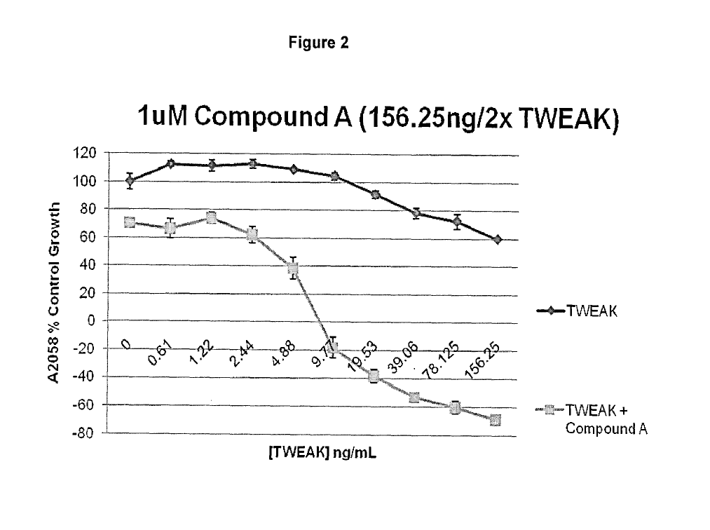

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated in FIG. 2.

### EXAMPLE 3

Treatment of A2058 melanoma cell with LIGHT in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and LIGHT. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted LIGHT at a final concentration of 400 ng, 80 ng, 16 ng, 3.2 ng, 640 pg, 128 pg, 25.6 pg, 5.12 pg, 1.024 pg, and one untreated well. Remaining cells are treated with 10 uL of serially diluted LIGHT and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

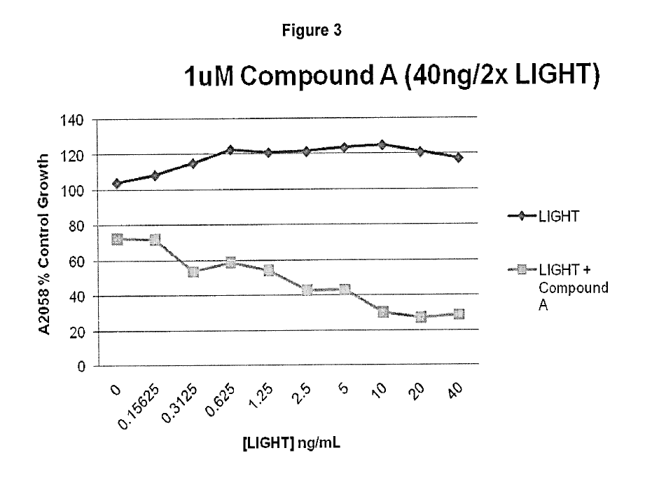

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated in FIG. 3.

### EXAMPLE 4

Treatment of A2058 melanoma cell with Fas in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and Fas. Treatments are done in triplicate. In plate one, cells are first treated with 10 ul of Compound A at a final concentration of 1 uM. Compound A is diluted in DMSO and then in media giving 0.2% final concentration of DMSO in treated wells. Cells are then treated with 10 uL of serially diluted Fas at a final concentration of 30 ng, 15 ng, 7.5 ng,3.75 ng, 1.875 ng, 937.5 pg, 468.75 pg, 234.375 pg, 117.2 pg, and one untreated well. Remaining cells are treated with 10 uL of serially diluted Fas and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

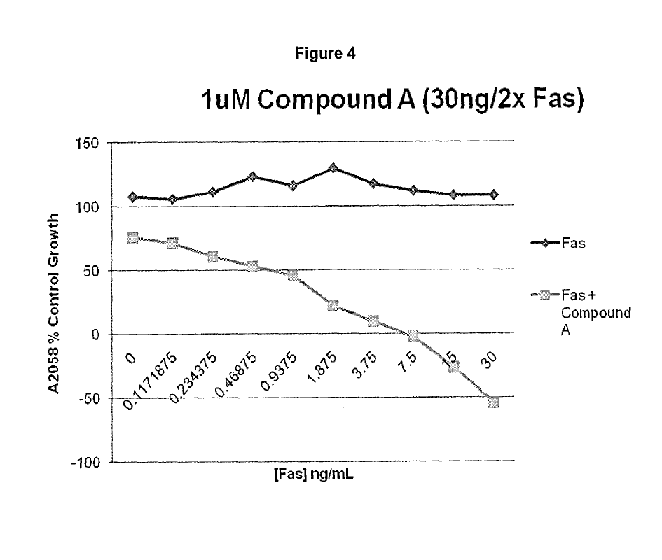

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated in FIG. 4.

### EXAMPLE 5

Treatment of A2058 melanoma cell with IL-1B in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and IL-1B. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted 1L-1B at a final concentration of 10 ng, 2 ng, 0.4 ng, 80 pg, 16 pg, 3.2 pg, 0.64 pg, 0.128 pg, 0.0256 pg, and one untreated well. Remaining cells are treated with 10 uL of serially diluted IL-1B and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis.

### EXAMPLE 6

Treatment of A2058 melanoma cell with TRAIL in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and TRAIL. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted TRAIL at a final concentration of 30 ng, 15 ng, 7.5 ng,3.75 g, 1.875 ng, 937.5 pg, 468.75 pg, 234.375 pg, 117.2 pg, and one untreated well. Remaining cells are treated with 10 uL of serially diluted TRAIL and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

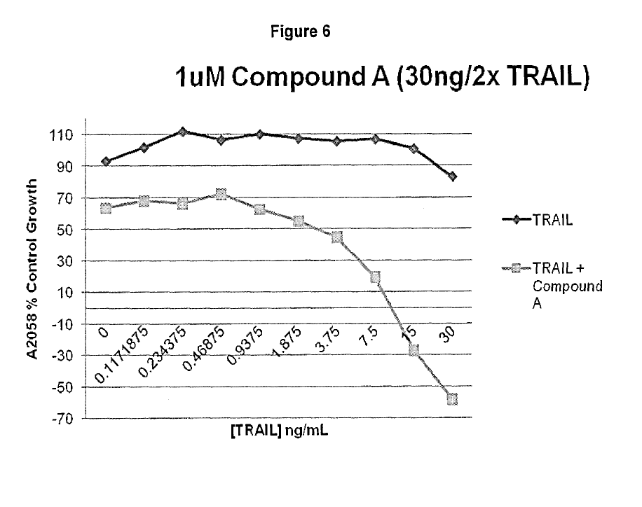

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated in FIG. 6.

### EXAMPLE 7

Treatment of A2058 melanoma cell with TNF alpha in the presence and absence of Compound A on cellular proliferation.

On day one, A2058 adherent melanoma cells are plated into two 96-well, clear, flat bottom plates. All wells in row A contain 90 uL of media. All wells in rows B-G contain 3000 cells per well in 90 uL of media. Plates are then incubated overnight for 18 hours at 37° C., 5% CO₂.

On day two, cells are treated with Compound A and TNF-α. Treatments are done in triplicate. In plate one, cells are first treated with 10 uL of Compound A at a final concentration of 1 uM. Compound A is diluted in DMSO and then in media giving 0.2% final concentration of DMSO in treated wells. Cells are then treated with 10 uL of serially diluted TNF-α at a final concentration of 20 ng, 4 ng, 0.8 ng, 0.16 ng, 32 pg, 6.4 pg, 1.28 pg, 0.256 pg, 0.0512 pg, and one untreated well. Remaining cells are treated with 10 uL of serially diluted TNF-α and no Compound A. Plate two is used as a time zero plate. To measure cell viability 50 uL of Cell Titer Glo (CTG) is added to row A, media only and B, cells and media. CTG measures the amount of ATP released from viable cells that can be measured by a luminescent reader. Cells with CTG are incubated for ten minutes at room temperature and then read.

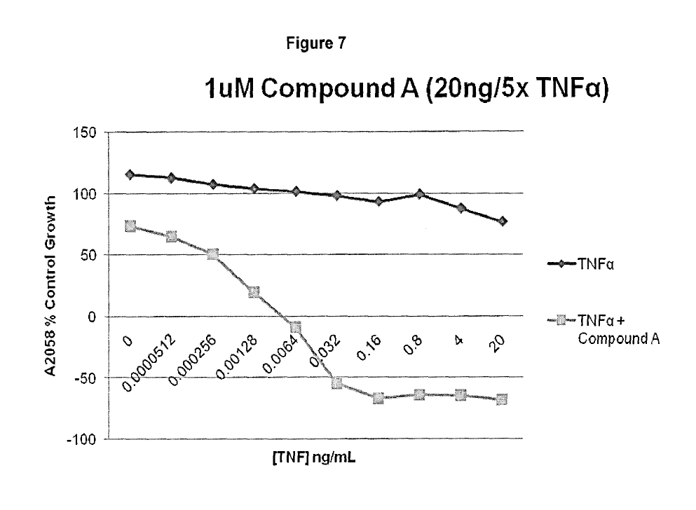

On day five, 50 uL of CTG is added to plate one, rows A-G, incubated for 10 minutes at room temperature and read on a luminescent program. Raw data is adjusted to account for the time zero plate as well as background noise. Triplicate values are averaged and percent control growth is calculated. Data is represented by line graph with concentration of cytokine on the x axis and percent control growth on the y axis as illustrated Figure 7.

### EXAMPLE 8

Compound A induced TNF-alpha in sensitive but not insensitive cancer cell lines.

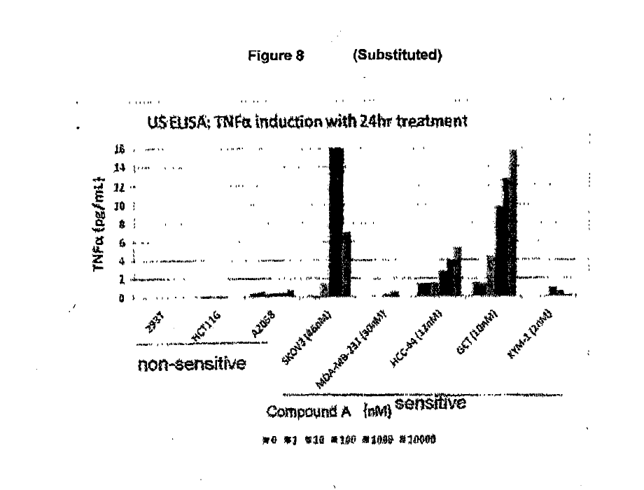

To test whether the engagement of the TNFα signaling pathway is requisite for single agent activity in vitro, a panel of cancer cell lines is evaluated for sensitivity to Compound A while in parallel, secreted TNFα expression levels are assessed by ELISA before and 24 hours after compound addition. Cell lines that do not respond to Compound A (up to 10 uM concentration) in 3-day viability assays are labeled as non-sensitive. Consistently, cell lines that up-regulated TNFα following Compound A treatment are more sensitive (as assessed in 3-day viability assays, IC50s are reported in FIG. 8) to Compound A than cell lines which do not (FIG. 8). Increased basal TNFα levels also positively correlate with sensitivity to Compound A (FIG. 8).

TNFα ELISA Method:

On day one, cancer cells are plated into two 96-well, clear, flat bottom plates. All wells in row A contain 200 uL of media. All wells in rows B-G contain 5000 cells per well in 180 ul of RPMI media with 10% FBS. Plates are then incubated overnight for 18 hours at 37 C, 5% CO₂. On day two, cells are treated with 20 uL of serially diluted Compound A. Compound A is diluted in DMSO and then in media giving 0.2% final concentration of DMSO. Control wells are treated with DMSO and no compound. Plate two is treated in parallel and used to measure viability. After incubating cells with compound for 24 hrs, 200 uL of media is removed from all treated wells and transferred to a new 96-well, clear, flat bottom plate. TNFα levels in the media are measured using the R&D Quantikine Human TNFα High Sensitivity ELISA assay, catalog number HSTA00D. Reagents provided in the kit are prepared as indicated and the provided assay protocol is followed as directed. The colorimetric readout is measured on a spectrometer at 490nM within 30 minutes of adding the stop solution. Raw data is normalized to account for background noise, duplicate values are averaged and concentrations are calculated from a standard curve (FIG. 8). To measure cell viability 50 uL of media is removed from treated wells in plate two and 50 uL of Cell Titer Glo (CTG) is added. CTG measures the amount of ATP released from viable cells that can be measured by a luminescent plate reader. Cells with CTG are incubated for ten minutes at room temperature and read on a luminescent plate reader. Raw data is normalized to account for background noise, duplicate values are averaged and percent control growth is calculated. These values are used to normalize the corresponding TNFα ELISA values reported in FIG. 8.

3-Day Viability Assay Method for IC50 Determination:

On day one, cancer cell lines are plated into two 96-well, clear, flat bottom plates. All wells in row A contain 200 uL of media. All wells in rows B-G contain 5000 cells per well in 180 ul of media. Plates are then incubated overnight for 18 hours at 37 C, 5% CO₂. On day two, cells are treated with serially diluted Compound A. Compound A is diluted in DMSO and then in media giving 0.2% final concentration of DMSO. Control wells are treated with DMSO and no compound. Plate two is used as the time zero plate. To measure cell viability 50 uL of media is removed from all wells and 50 uL of Cell Titer Glo (CTG) is added to row A, media only, and B, cells and media. Cells with CTG are incubated for ten minutes at room temperature and then read. CTG measures the amount of ATP released from viable cells that can be measured by a luminescent plate reader. On day five, 50 uL of media is removed from all wells and 50 uL of CTG is added to plate one, rows A-G, incubated for 10 minutes at room temperature and read on a luminescent plate reader. Raw data is normalized to account for the time zero plate as well as background noise. Triplicate values are averaged and percent control growth is calculated. The dose of Compound A required to inhibit growth of the cancer lines by 50% relative to vehicle treated control cells is determined and reported in FIG. 8.

### EXAMPLE 9

RelA but not RelB is required for Compound A-induced TNFα

By stimulating the autoubiquitination and proteosome mediated degradation of CIAP1, Smac mimetics hypersensitize tumor cells to TNFα-mediated apoptosis. Indeed, Smac mimetics induce the production of TNFα in sensitive but not insensitive tumor cell lines. The precise mechanism of NFkB activation and subsequent TNFα induction is not well understood, and identifying the mechanism by which Compound A induces TNFα provides potential biomarkers for patient stratification.

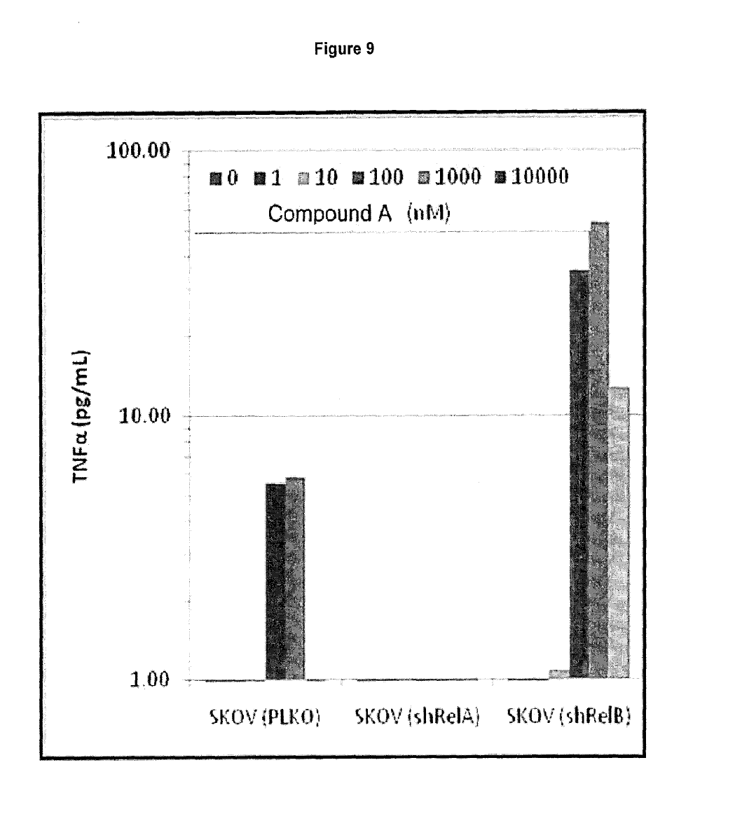

To elucidate this mechanism, nodes of the canonical and non-canonical NFkB pathways and are knocked down and induction of TNFα expression, a marker of NFkB activation, after treatment with the Smac mimetic compound Compound A, is assessed. In the canonical NFkB pathway, shRNA-mediated knockdown of RelA in the SK-OV-3 ovarian carcinoma cell line (SKOV) ablated TNF induction (as measured by ELISA, FIG. 9), suggesting that activation of the canonical pathway is required for Compound A activity. In the non-canonical NFkB pathway, shRNA-mediated knockdown of RelB does not impact Compound A-mediated induction of TNFα (FIG. 9). Overall, these results indicate that induction of TNFα by Compound A requires canonical NFkB signaling. Therefore, tumors with active or functional canonical NFkB signaling, as evidenced by TNFα expression or other markers of active NFkB signaling, are more likely to respond to Compound A.

TNFα ELISA Method:

On day one, SKOV cancer cells are plated into two 96-well, clear, flat bottom plates. All wells in row A contain 200 uL of media. All wells in rows B-G contain 5000 cells per well in 180 ul of RPMI media with 10% FBS and 1 ug/mL of puromycin. Plates are then incubated overnight for 18 hours at 37 C, 5% CO2. On day two, cells are treated with 20 uL of serially diluted Compound A. Compound A is diluted in DMSO and then in media giving 0.2% final concentration of DMSO. Control wells are treated with DMSO and no compound. Plate two is treated in parallel and used to measure viability. After incubating cells with compound for 24 hrs, 200 uL of media is removed from all treated wells and transferred to a new 96-well, clear, flat bottom plate. TNFα levels in the media are measured using the R&D Quantikine Human TNFalpha High Sensitivity ELISA assay, catalog number HSTA00D. Reagents provided in the kit are prepared as indicated and the provided assay protocol is followed as directed. The colorimetric readout is measured on a spectrometer at 490 nM within 30 minutes of adding the stop solution. Raw data is normalized to account for background noise, duplicate values are averaged and concentrations are calculated from a standard curve (FIG. 9). To measure cell viability 50 uL of media is removed from treated wells in plate two and 50 uL of Cell Titer Glo (CTG) is added. CTG measures the amount of ATP released from viable cells that can be measured by a luminescent plate reader. Cells with CTG are incubated for ten minutes at room temperature and read on a luminescent plate reader. Raw data is normalized to account for background noise, duplicate values are averaged and percent control growth is calculated. These values are used to normalize the corresponding TNFα ELISA values reported in Figure 9.

### EXAMPLE 10

Compound A Treatment Induced TNFα in Breast Cancer Tumor Cell Line Xenografts

In in vitro assays, Compound A induces the induction of TNFα, activation of caspases and subsequent cell death in a variety of tumor cell lines, including the MDA-MB-231 breast cancer cell line. To assess whether the induction of TNFα observed in the MDA-MB-231 breast tumor cell line translated to an in vivo setting, an experiment is performed in mice harboring orthotopically implanted MBA-MD-231 tumors.

A single oral dose of Compound A is administered 39 days following tumor cell implantation when mean tumor volumes reached approximately 141 mm3. Tumors are harvested from each animal, lysed, and analyzed by ELISA to determine TNFα concentrations.

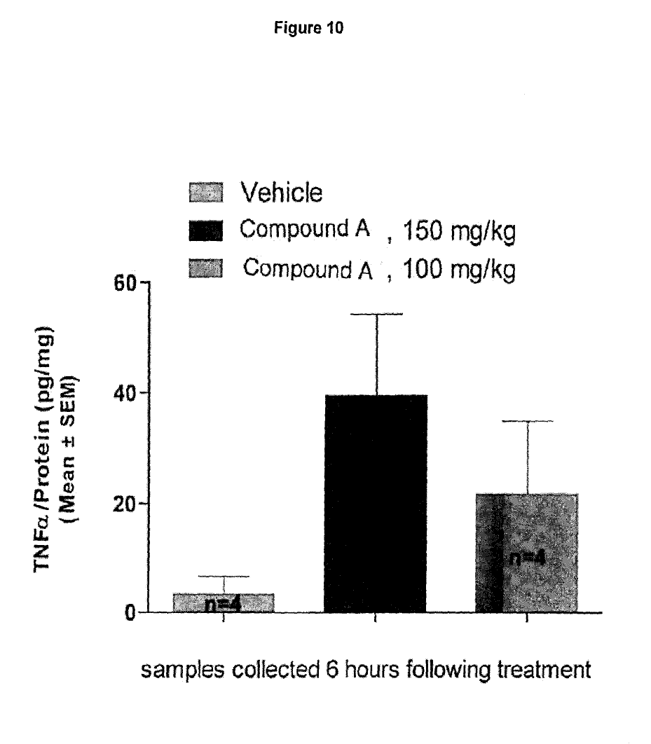

Following a single oral dose of Compound A, there is an approximate 10-fold increase in TNFα in MDA-MB-231 tumor lysates (FIG. 10).

### EXAMPLE 11

TNFα expression is correlated with response to Compound A in primary human breast and lung tumor xenografts.

Patient-derived xenograft models in which human tumors are surgically resected then directly implanted and subsequently passaged in nude mice are potentially more clinically relevant than cell line models since there is no selective pressure for two dimensional growth on a plastic stratum.

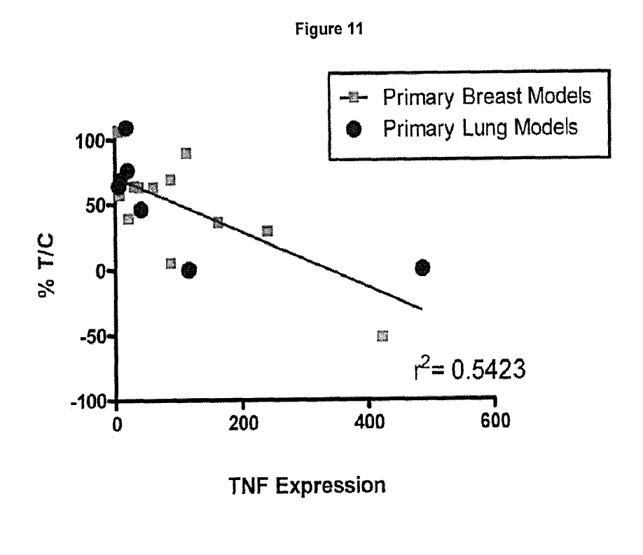

Compound A has been tested for single agent activity in 18 patient-derived tumor models representing both triple negative breast cancer and Non Small Cell Lung Carcinoma (NSCLC) models. Tumor growth inhibition by Compound A is reported as % T/C (percent growth of tumors in mice treated with Compound A relative to the growth of tumors in Control vehicle treated mice). Complete regression of a tumor would be reported as −100% T/C, tumor stasis (no growth) would be reported as 0% T/C, and no effect would be reported as 100% T/C. A range of responses have been observed for Compound A from tumor regression to no effect. Stasis (T/C≦40%) has been observed in 25% of primary models assessed. In agreement with the observation that tumor cell lines exhibiting sensitivity to Compound A in vitro are characterized by autocrine TNF signaling, the most sensitive primary tumor models also exhibited high TNFα expression based on Affymetrix mRNA transcription profiling data (FIG. 11).

Variations, modification, and other implementations of what is described herein will occur to those of ordinary skill in the art without departing from the spirit and the essential characteristics of the present teachings. Accordingly the scope of the disclosure is to be defined not by the preceding illustrative description but instead by the following claims, and all changes that come within the meaning and range of equivalency of the claims are intended to be embraced therein.

1. A method to predict which patients will respond to an IAP inhibiting compound comprising:
a) administering an IAP inhibitor compound to a patient, and
b) measuring IL1B, Lymphotoxin alpha (LTa), TWEAK, LIGHT, Fas, TNF alpha or TRAIL levels.

2. The method of claim 1, wherein the IAP inhibiting compound has the structure of formula I: 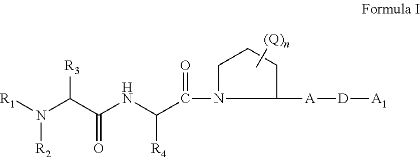
or pharmaceutically acceptable salts thereof, wherein
R₁ is H, C₁-C₄ alkyl, C₂-C₄ alkenyl, C₂-C₄ alkynyl or C₃-C₁₀ cycloalkyl, which R₁ may be unsubstituted or substituted;
R₂is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂-Z or R₂ and R₃ taken together with the nitrogen atom to which they are attached forma heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C (O) C₀-C₁₀ arylalkyl OC₀-C₁₀ arylalkyl or S(O)r C₀-C₁₀ arylalkyl, which alkyl and aryl groups may be unsubstituted or substituted;
r is 0,1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆-—C₃-C₇ cycloalkyl, aryl, aryl C₁-C₁₀ alkyl, O—(CH₂)₀₋₆ aryl, (CH₂) ₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3; 4, 5, 6 or 7;
het is a 5-7 membered monocyclic heterocyclic ring containing 1 -4 heteroring atoms selected from N, O and S or an 8-12 membered fused ring system that includes one 5-7 membered monocyclic heterocyclic ring containing 1, 2, or 3 heteroring atoms selected from N; O and S, which het is unsubstituted or substituted;
R₁₁ and R₁₂ are independently H, C₁-C₁₀ alkyl, (CH₂)₀₋₆—C₃C₇cloalkyl, (CH₂)₀₋₆—(CH)₀₋₁(aryl)₁₋₂, C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)₁₁, R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ area substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S-C₁-C₆alkyl, CF₃ or NR₁₁R₁₂;
substituted cycloalkyl substituents of R₁₁ and R₁₂ are substituted by one or more substituents selected from C₂-C₁₀ alkene; C₁-C₆alkyl; halogen; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, hitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein; the substituents on R₁, R₂, R₃, R₄, Q; and A and A₁ groups are independently halo, hydroxy,,lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy; lower alkanoyl, aryloyl lower arylalkanoyl, carbamoyl; N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate; sulfanyl lower alkyl, aryl sulfonamide; halogen substituted aryl sulfonate, lower alkylsulfinyl, arylsuffinyl, aryl-lower alkylsuffinyl, lower alkylarylsulfinyl, lower alkylsulfonyl, arylsulfonyl, aryl-lower alkylsulfonyl, lower arylalkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl; halo, lower alkenyl, lower alkynyl, hydroxy, lower alkoxy, nitro, amino, lower alkyl, amino, diloweralkyl amino; cyano, carboxy, lower carbalkoxy, formyl, lower alkanoyl, oxo, carbarmoyl, N-lower or N, N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

3. A method for determining the responsiveness of an individual with a disease characterized by IL1B, Lymphotoxin alpha (LTa), TWEAK, LIGHT, Fas, TNF alpha or TRAIL signaling to treatment with a IAP inhibiting compound comprising:
a) administering an IAP inhibitor compound to a patient, and
b) measuring IL1B, Lymphotoxin alpha (LTa), TWEAK, LIGHT, Fas, TNF alpha or TRAIL levels.

4. A method for treating diseases characterized by IL1B, Lymphotoxin alpha (LTa), TWEAK, LIGHT, Fas, TNF alpha or TRAIL signaling comprising:
a) administering an IAP inhibitor compound, and
b) measuring IL1B, Lymphotoxin alpha (LTa), TWEAK, LIGHT, Fas, TNF alpha or TRAIL levels.

5. The method of claim 3 wherein the IAP inhibiting compound has the structure of formula I: 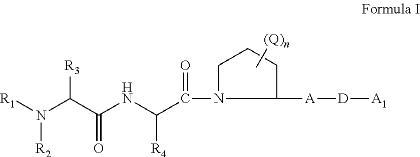
or pharmaceutically acceptable salts thereof, wherein
R₁ is H, C₁-C₄ alkyl, C₂-C₄ alkenyl, C₂-C₄ alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C (O) C₀-C₁₀ arylalkyl OC₀-C₁₀ arylalkyl, or S(O)r C₀-C₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆-C₃-C₇ cycloalkyl, aryl, aryl C₁-C₁₀ alkyl, O—(CH₂)₀₋₆ aryl, (CH₂) ₁₋₆het, het, O—(CH2)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are-unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, Which het is unsubstituted or substituted;
R₁₁ and R₁₂ are independently H, C₁-C₁₀ alkyl, (CH₂)₀₋₆—C₃-C₇cycloalkyl, (CH₂)₀₋₆-O-fluorenyl, C(O)—NH—(CH₂)₀₋₆-aryl, C(O)—(CH₂)₀₋₆-aryl, C(O)—(CH₂)₁₋₈-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)ₘNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;
substituted cycloalkyl substituents of R₁₁ and R₁₂ are substituted by one or more substituents selected from a C₂-C₁₀alkene; ₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide; halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino; lower alkyl, amino, diloweralkyl amino, cyano, carboxy, lower carbalkoxy, formyl, lower alkanoyl, oxo, carbarmoyl, N-lower or N, N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

6. The method according to claim 1 wherein where the IAP inhibitor compound is selected from N-1-Cyclohexyl-2-{2-[4-(4-fluoro-benzoyl)-thiazol-2-yl]-pyrrolidin-1-yl}-2-oxo-ethyl)-2-methylamino-propionamide; N-[Cyclohexyl-(ethyl-{1-[5-(4-fluoro-benzoyl)-pyridin-3-yl]-propyl}carbamoyl)-methyl]-2-methylamino-propionamide; N-(1-Cyclohexyl-2-{2-[5-(4-fluoro-phenoxy)-pyridin-3-yl]-pyrrolidin-1-yl}-2-oxo-ethyl)-2-methylamino-propionamide; and N-[1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

7. A method of using IAP inhibitor compounds in the treatment of proliferative diseases characterized by IL1B, Lymphotoxin alpha (LTa), TWEAK, LIGHT, Fas, TNF alpha or TRAIL signaling;

8. A method of using a compound of the formula I, or an N-oxide or pharmaceutically acceptable salt thereof, in the treatment of a disease characterized by IL1B; Lymphotoxin alpha (LTa), TWEAK, LIGHT, Fas, TNF alpha or TRAIL signaling wherein the compound of formula I has the following structure: 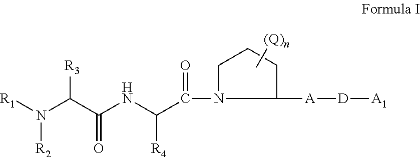
or pharmaceutically acceptable salts thereof, wherein
R₁is H, C₁-C₄ alkyl, C₂-C₄ alkenyl, C₂-C₄ alkynyl or C₃-C₁₀ cycloalkyl, which R₁ may be unsubstituted or substituted;
R₂ is H, C₁-C₄ alkyl; C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)r, C(O)—C₁-C₁₀ alkyl, O—C₁-C₁₀ alkyl, S(O)r—C₁C₁₀ alkyl, C (O) C₀-C₁₀ arylalkyl OC₀-C₁₀ arylalkyl, or S(O)r C₀-C₁₀ arylalkyl, which alkyl and aryl groups,may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂) ₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1,2, or 3 heteroring atoms selected from N, O and S, which het is unsubstituted of substituted;
R₁₁ and R₁₂ are independently H, C₁-C₁₀ alkyl, (CH₂)₀₋₆—C₃-C₇cloalkyl, (CH₂)₀₋₆—(CH)₀₋₁(aryl₁₋₂, C(O)—C₁-C₁₀alkyl, —C(O)—(CH₂₁₋₆—C₃-C₇cycloalkyl, —C(O)—O—(CH₂)₀₋₆-aryl), —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆-C₃-C₇cycloalkyl, —C(S)—O—(CH₂)₀₋₆-aryl, —C(S)—(CH₂)₀₋₆—O-fluorenyl, C(S)—NH—(CH₂aryl, —C(S)—(CH₂)₀₋₆-aryl or C(S)—CH₂)₁₋₆-het, C(O)R₁₁, C(O)NR₁₁R₁₂, C(O)OR₁₁, S(O)nR₁₁, S(O)ₘNR₁₁R₁₂, m=1 or2, C(S)R₁₁ C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₂alkyl, halogen, OH, O—C₁-C₆alkyl; —S—C₆alkyl, CF₃ or NR₁₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alky), carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl; aryl sulfonamide, halogen substituted aryl sulfonate, lower alkylsulfinyl; arylsuffinyl; aryl-lower alkylsulfinyl, lower alkylarylsulfinyl, lower alkylsulfonyl, arylsurfonyl, an/Mower alkylsulfonyl, lower aryl alkyl lower alkylarylsurfonyl, halogen-lower alkylmercapto, halogen-lower alkylsulfonyl, phosphono (—P(═O)(OH)₂), hydroxy-lower alkoxy phosphoryl or di-lower alkoxyphosphpryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen; oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkylamino, cyano, carboxy, lower carbalkoxy, formyl, lower alkanoyl, oxo, carbarmoyl, N-lower or N, N-dilower alkyl carbamoyl, mercapto, or lower alkylthio, and
R₉, R₁₀, and R₁₃ are independently hydrogen, lower alkyl, halogen substituted, lower alkyl, aryl, aryl lower alkyl, halogen substituted aryl, halogen substituted aryl lower alkyl.

9. The method of using a compound of the formula I, according to claim 9, or a pharmaceutically acceptable salt thereof, for the manufacture of a pharmaceutical composition for the treatment of a disease characterized by IL1B, Lymphotoxin alpha (LTa), TWEAK, LIGHT, Fas, TNF alpha or TRAIL signaling.

10. The method of treatment a disease characterized by IL1B, Lymphotoxin alpha (LTa), TWEAK, LIGHT, Fas, TNF alpha or TRAIL signaling, comprising administering to a warm-blooded animal, in need of such treatment a pharmaceutically effective amount of a compound of the formula I, or a pharmaceutically acceptable salt thereof, according to claim 9.

11. The method according to claim 9 where the compound of formula I is selected from N-1-Cyclohexyl-2-{2-[4-(4-fluoro-benzoyl)-thiazol-2-yl]-pyrrolidin-1-yl}-2-oxo-ethyl)-2-methylamino-propionamide; N-[Cyclohexyl-(ethyl-{1-[5-(4-fluoro-benzoyl)-pyridin-3-yl]-propyl}carbamoyl)-methyl]-2-methylamin-propionamide; N-(1-Cyclohexyl-2-{2-[5-(4-fluoro-phenoxy)-pyridin-3-yl]-pyrrolidin-1-yl}-2-oxo-ethyl)-2-methylamino-propionamide; and N-[1-Cyclohexyl-2-(2-{2-[(4-fluorophenyl)-methyl-amino]-pyridin-4-yl}pyrrolidin-1-yl)-2-oxo-ethyl]-2-methyalamino-propinamide and pharmaceutically acceptable salts thereof.

12. The method according to claim 10 where the compound of formula I is selected from N-(1-Cyclohexyl-2-{2-[4-(4-fluoro-benzoyl)-thiazol-2-yl]-pyrrolidin-1-yl}-2-oxo-ethyl)-2-methylamino-propionamide; N-[Cyclohexyl-(ethyl-{1-[5-(4-fluoro-benzoyl)-pyridin-3-yl]-propyl}carbamoyl)-methyl]-2-methylamino-propionamide; N-(1-Cyclohexyl-2-{2-[5-(4-fluoro-phenoxy)-pyridin-3-yl]-pyrrolidin-1-yl}-2-oxo-ethyl)-2-methylamino-propionamide; and N-[1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

13. A The method according to claim 11 where the compound of formula I is selected from N-(1-Cyclohexyl-2-{2-[4-(4-fluoro-benzoyl)-thiazol-2-yl]-pyrrolidin-1-yl}-2-oxo-ethy)-2-methylamino-propionamide; N-[Cyclohexyl-(ethyl-{1-[5-(4-fluoro-benzoyl)-pyridin-3-yl]-propyl}carbamoyl)-methyl]-2-methylamino-propipnamide; N-(1-Cyclohexyl-2-{2-[5-(4-fluoro-phenoxy)-pyridin-3-yl]pyrrolidin-1-yl}-2-oxo-ethyl-2-methylamino-propinamide and pharmaceutically acceptable salts thereof.

14. The method according to claim 9 wherein the disease is a proliferative disease.

15. The method according to claim 9 wherein the disease is a selected from cancers, such as solid tumors and blood-born tumors; heart disease,, such as congestive heart failure; and viral, genetic; inflammatory, allergic, and autoimmune diseases.
